(19) United States
(12) Patent Application Publication
(10) Pub. No.: US 2007/0110689 A1
(43) Pub. Date: May 17, 2007

(54) Oral anaesthetic gel

(76) Inventor: Mark Feldschuh, Seaford (AU)

Correspondence Address: EDWIN D. SCHINDLER, FIVE HIRSCH AVENUE, P.O. BOX 966, CORAM, NY, 11727-0966, US

(21) Appl. No.: 10/597,208

(22) PCT Filed: Dec. 22, 2004
(86) PCT No.: PCT/AU04/01817

§ 371 Date: Jul. 16, 2006

(30) Foreign Application Priority Data
Jan. 15, 2004 (AU) 2004900176

(51) Int. Cl. A61K 8/00

(52) U.S. Cl. 424/70.1

(57) ABSTRACT

A gel-based anaesthetic, which is also palatable and can be used on mucosal surfaces, includes an anaesthetic in a transdermal gel-base having added flavoring with a bitterness suppressant. The gel base is preferably pluronic gel and the anaesthetic is preferably lignocaine.

### AREA OF THE INVENTION

This invention relates to the area of topical anaesthesia or desensitisation. In particular it relates to a topical anaesthetic which is adapted to be used on mucous membranes and can be usefully applied in the field of dentistry despite having other applications.

### BACKGROUND TO THE INVENTION

Local anaesthetics have been used in creams and ointments for many years. Usually however there is a problem with the penetration of the drug or chemical due to the physio-chemical properties of both the drug and the base in which it is used.

In addition one of the products used topically on non mucous membranes is a cream and is not suitable for use on mucous membranes such as in the mouth. Another product which can be used orally is a paste that has been formulated for the mouth but unfortunately does not adhere to the gum or mouth particularly well when used for dental purposes.

It has been suggested that a gel base could be used to adhere in the mouth however to date none exist which are able to provide the anaesthetic effect required and additionally are quite unpalatable and therefore unsuitable for oral anaesthetic use.

### OUTLINE OF THE INVENTION

It is an object of this invention to provide a topical anaesthetic for use on mucosal surfaces which does not exhibit the problems outlined above. It is a further object of the invention to provide such a topical anaesthetic which is sufficiently palatable that it can readily be used for dental purposes.

The invention is an oral anaesthetic gel including an anaesthetic in a transdermal gel base having added flavouring with a bitterness suppressant.

It is preferred that the gel base used be Pluronic Lecithin Organogel (PLO or Pluronic Gel) and that its viscosity be adjusted as required by the addition of suitable thickeners. It is further preferred that PLO strengths range from 2% to 20%.

It is further preferred that the active agent or ingredient, otherwise referred to as the active pharmaceutical ingredient (API) be lignocaine base USP or alternatively the HCL salt. It is also preferred that other active ingredients may be tetracaine benzocaine, amethocaine or prilocalne as salts.

In order that the invention may be more readily understood we shall describe by way of non limiting example a particular embodiment of the invention.

### EXAMPLES OF EMBODIMENTS OF THE INVENTION

A first preferred application of the invention is in the area of dentistry and will be described here.

This embodiment of the invention is a gel formulation that is very quickly absorbed into the mucosa. As absorption is rapid the dentist can inject anaesthetic into a patient's gum with a major reduction in pain in the injection site in as little as 30 seconds or up to 2 minutes.

Trauma is further reduced psychologically by the presence of palatable flavouring in the gel which masks the customary bitter taste of the analgesic material used as the anaesthetic.

Examples of the invention to be described here are PLO gel formulations having Lignocaine and being flavoured.

Dental Anaesthetic Gel

A preferred formulation for a dental anaesthetic gel is as follows:

Lignocaine USP 10.0 g

Sodium Metabisulphite 0.1 g

Ethoxydiglycol Reageant 10 ml

Lecithin Isopropyl Palmitate/Myristate Solution 22 ml

Flavouring 12 ml

Saccharin Sodium 0.20 g

Stevia powder extract 1 g

Simethicone 0.02 ml

Pluronic Gel 20% up to 100 ml.

The procedure for making the formulation is as follows:
Calibrate a beaker to final volume
Weigh the powder ingredients
Add Lignocaine, Saccharine, Sodium Metabisulphate to the beaker with flavouring and ethoxy diglycol reagent.
Add a magnetic stirring bar and stir mixture well
Create a vortex with the stir bar and slowly add the stevia to avoid lumps.
Add lecithin isopropyl myristate solution and allow lignocaine to dissolve
remove stirring bar and add Pluronic gel 20% to volume
pour mixture into an appropriately sized unguator jar, remove excess
Close lid tightly with mixing blade in place, expel all air and mix for a few minutes

If desired the gel may be stored in a syringe with any excess air removed or otherwise stored as desired. The air removal is preferably achieved by turning the syringe upside down and allowing the gel to settle on the plunger before removing the air.

The compounding procedure is an important part of the process as force used in mixing can encourage micelle formation. It is therefore preferred that this be reduced by using syringe to syringe techniques, a Dremel tool with mixing blade, electric mortars and ointment mills which can aid in the process.

Teething Gel

A preferred formulation for a teething gel is as follows:

Lignocaine USP 2.0 g

Chlorhexadine Acetate 5% Solution 0.7 ml

Phenylepherine HCL USP 0.25 g

Sodium Metabisulphate 0.1 g

Ethoxy Diglycol Reagent 10 m,

Lecithin Isopropyl Palmitate/Myristate Solution 22 ml

Flavouring 12 ml

Pluronic Gel 20% up to 100 ml.

The procedure for making the formulation is as follows:
Weigh the Lignocaine, Phenylepherine HCl, Sodium Metabisulphate and add to an appropriately calibrated beaker.
Measure the Ethoxy Diglycol Reagent, and Lecithin Isopropyl Palmitate Solution, Chlorhexidine solution and add to the beaker with a magnetic stirring bar.
place beaker on a stirring plate and stir until the ingredients are mixed
whilst stirring add flavouring, sweetener, bitterness suppressant and colour if necessary
remove stirring bar and add Pluronic gel 20% to volume
pour mixture into an appropriately sized unguator jar, remove excess
Close lid tightly with mixing blade in place, expel all air and mix for a few minutes

If desired the gel may be stored in a syringe with any excess air removed or otherwise stored as desired. The air removal is preferably achieved by turning the syringe upside down and allowing the gel to settle on the plunger before removing the air.

The compounding procedure is an important part of the process as force used in mixing can encourage micelle formation. It is therefore preferred that this be reduced by using syringe to syringe techniques, a Dremel tool with mixing blade, electric mortars and ointment mills which can aid in the process.

While a variety of flavours may be used they may include the following:

PINA COLADA per 100 ml

Bitterness Suppressant 2.5 ml, Pineapple 3 ml, Pina Colada 2.5 ml, Peach Oil 0.5 ml, Coconut 1 ml, yellow 0.2 ml

CARAMEL per 100 ml

Bitterness Suppressant 2.5 ml, Cinnamon Oil 1 ml, Caramel 8 ml

STRAWBERRY per 100 ml

Bitterness suppressant 2.5 ml, Strawberry 4 ml, Blackberry Oil 1 ml, watermelon 2.5 ml, Krisgel to thicken, red 0.1 ml

ORANGE per 100 ml

Bitterness suppressant 2.5 ml, Orange cream 3.5 ml, Orange natural concentrate 3.5 ml, Tangerine oil 1 ml, red 0.1 ml, yellow 0.1 ml

BUBBLEGUM per 100 ml

70 drp sweet, 50 drp bitter, 70 drp bubble, 60 drp banana, 40 drp grape

TROPICAL FLAVOUR per 100 ml

90 drp sweet, 50 drp bitter, 80 drp strawberry, 20 drp peach oil, 10 drp pineapple, 40 drp pina colada, 10 drp coconut, 10 drp banana

WILD BERRY per 100 ml

90 drp sweet, 50 drp bitter, 80 drp strawberry, 20 drp blackberry oil, 40 drp watermelon and 2% Krisgel to thicken.

The above are examples of the gel formulation of the invention and it is envisaged that actual concentrations of ingredients can vary as can the actual ingredients which are chosen depending on the specific application.

For example the strength of the analgesic used in the gel for dentistry could be typically up to 10% lignocaine as described above while for over the counter type medications such as the teething gel 1% or 2% could be used.

In addition the previously suggested anaesthetic agents can generally be used up to 10% to achieve a specified effect while benzocaine can be up to 20%.

As has also been suggested the viscosity of any batch of the gel formulation can be adjusted by adding an appropriate thickener.

Clearly the concept of can be achieved in a variety of ways and while particular embodiments of the invention have been described herein it is to be understood that variations and modifications in the features described can still lie within the scope of the invention.

1-6. (canceled)

7. An oral anaesthetic gel, comprising:
a transdermal gel base;
an anaesthetic in said transdermal gel base; and,
a flavoring with a bitter suppressant added to said transdermal gel base.

8. The oral anaesthetic gel according to claim 7, wherein said transdermal gel base is pluronic gel.

9. The oral anaesthetic gel according to claim 7, wherein said anaesthetic is lignocaine.

10. The oral anaestheic gel according to claim 7, wherein said anaesthetic is lignocaine, said transdermal gel base is pluronic gel, and further comprising chlorhexidine acetate, phenylephrine HCl, sodium meta-bisulfite, ethoxyl diglycol reagent, lecithin and isopropyl palmate/myristate solution.

11. The oral anaestheic gel according to claim 10, wherein said anaesthetic is lignocaine USP 2 g, said transdermal gel base is pluronic gel in an amount of up to, and including, 100 ml, and further comprising chlorhexidine acetate 5% solution 0.7 ml, phenylephrine HCl USP 0.25 g, sodium meta-bisulfate 0.1 g, ethoxyl diglycol reagent 10 ml, lecithin isopropyl palmate/myristate solution and said flavoring in an amount of 12 ml.

12. The oral anaestheic gel according to claim 10, wherein said anaesthetic is lignocaine USP 10 g, said transdermal gel base is pluronic gel in an amount of up to, and including, 100 ml, and further comprising sodium meta-bisulfate 0.1 g, ethoxy diglycol reagent 10 ml, lecithin isopropyl palmitate/myristate solution 22, saccharin sodium 0.2 g, stevia powder extract 1 g, simethicone 0.02 ml and said flavoring in an amount of 12 ml.
